(19) United States
(12) Patent Application Publication
(10) Pub. No.: US 2004/0048821 A1
(43) Pub. Date: Mar. 11, 2004

(54) Enhancement of drug cytotoxicity in tumor cells containing mutant Rb gene

(75) Inventor: Scott W. Lowe, Cold Spring Harbor, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Cold Spring Harbor Laboratory, Cold Spring Harbor, NY (US)

(21) Appl. No.: 10/401,116

(22) Filed: Mar. 26, 2003

Related U.S. Application Data
(63) Continuation of application No. 09/103,953, filed on Jun. 24, 1998, now abandoned.
(60) Provisional application No. 60/051,086, filed on Jun. 27, 1997.

(51) Int. Cl. A61K 48/00; C07K 14/705; C07H 21/04

(52) U.S. Cl. 514/44; 536/23.2; 530/350

(57) ABSTRACT

Described herein are mutant forms of the adenovirus E1A oncoprotein which are unable to bind and inactivate retinoblastoma (Rb) protein and are defective in promoting apoptosis and chemosensitivity in normal (non-tumorigenic or nonmalignant) cells, but enhance apoptosis and sensitivity to toxic agents (e.g., chemotherapeutic agents, radiation) in Rb protein deficient mutant cells. Such E1A mutant oncoproteins are useful to enhance apoptosis and sensitivity to toxic agents in Rb protein deficient mammalian cells. Also described are agents, useful to promote apoptosis and chemosensitivity in Rb deficient cells, which mimic the activity of an E1A region involved in binding p300 and CBP proteins. Such E1A mimics are, for example, polypeptides which consist essentially of the amino acid residues of such an E1A region (e.g., the N-terminal region, CR1), DNA encoding the E1A region or small organic molecules which mimic the activity of the E1A region. The E1A region mimics are also the subject of the present invention, as are a method of enhancing apoptosis and sensitivity to chemotherapeutic agents and radiation in Rb deficient cells using the E1A mutants and/or mimics, a method of enhancing apoptosis and chemosensitivity and sensitivity to radiation in Rb deficient cells (e.g., tumor or malignant cells) in an individual who is being treated with chemotherapeutic agents and/or radiation using the E1A mutants and/or mimics, a method of identifying an E1A mutant and a method of identifying a molecule which mimics the function of an E1A mutant as described herein.

### RELATED APPLICATION(S)

This application is a continuation of U.S. application Ser. No. 09/103,953, filed Jun. 24, 1998, which claims the benefit of U.S. Provisional Application No. 60/051,086, filed Jun. 27, 1997. The entire teachings of the above application(s) are incorporated herein by reference.

### GOVERNMENT SUPPORT

Work described herein was funded by Grant Number 5P01 CA13106-25 and Grant Number 2P01 CA13106-26 from the National Cancer Institute. The United States Government has certain rights in the invention.

### BACKGROUND OF THE INVENTION

Cancer therapy is an area of medicine in which there is an ongoing need for more effective treatment, particularly drugs and protocols which have enhanced cytotoxicity toward tumor cells.

### SUMMARY OF THE INVENTION

Described herein are mutant forms of the adenovirus E1A oncoprotein which are unable to bind and inactivate retinoblastoma (Rb) protein and are defective in promoting apoptosis and chemosensitivity in normal (non-tumorigenic or nonmalignant) cells, but enhance apoptosis and sensitivity to toxic agents (e.g., chemotherapeutic agents, radiation) in Rb protein deficient mutant cells. Such E1A mutant oncoproteins are useful to enhance apoptosis and sensitivity to toxic agents in Rb protein deficient mammalian cells. Rb protein mutant or deficient cells include mammalian cells which lack a Rb gene or contain a mutant Rb gene and in which, as a result, Rb gene product is not produced or is produced to a lesser extent than in corresponding cells which contain a normal Rb gene and mammalian cells in which Rb gene product is produced but is inactivated, directly or indirectly. Rb protein mutant or deficient cells also include mammalian cells in which there are mutations or alterations in the pathway through which Rb acts, rendering Rb defective or inactive. Rb mutant cells are also referred to herein as Rb protein deficient cells; the two terms are used interchangeably. Also described is a method of enhancing drug cytotoxicity specifically in tumor cells which are Rb mutant cells. This method is useful in specifically enhancing the cytotoxicity of toxic agents, such as irradiation or chemotherapeutic agents (e.g., adriamycin), toward tumor cells and, thus, in the treatment of individuals receiving anti-cancer therapy. A particular advantage of the method is that Rb protein deficient cells (e.g., tumor cells) are killed to a greater extent than are normal cells and less drug is needed to kill tumor (Rb protein deficient) cells than is necessary using conventional methods of treatment.

In one embodiment of a method of enhancing the cytotoxic effects of an anti-cancer drug or irradiation, an E1A mutant (or mutants) which fails to inactivate Rb in normal cells is administered to an individual to whom one or more chemotherapeutic drugs and/or irradiation are also administered. The E1A mutant enters cells in the individual, including cells to be killed by the chemotherapeutic drug or irradiation, resulting in enhanced sensitivity of Rb deficient tumor cells, without a corresponding increased chemosensitivity of normal (Rb containing) cells. Such E1A mutants lack, for example, an internal region necessary for binding to and inactivation of Rb protein; in one instance the E1A mutant lacks amino acid residues from about 120 to 140 of E1A. Such E1A mutants also include those in which one or more amino acid residues in an internal region is deleted, modified or replaced by an amino acid other than that which normally occurs in E1A. In one embodiment, amino acid residues 47 and 124, which are tyrosine residues in E1A, are replaced by another amino acid residue, such as histidine residues.

Also described herein are E1A mutants which are defective in promoting apoptosis and chemosensitivity in both human and mouse cells and which are impaired in binding the p300 and/or CREB binding proteins (CBP). Examples of such mutants are E1A mutants which lack the E1A N-terminal region (amino acid residues 2 to 36) and E1A mutants which lack at least a portion of conserved region 1 (CR1).

Also described are agents, useful to promote apoptosis and chemosensitivity in Rb deficient cells, which mimic the activity of an E1A region involved in binding p300 and CBP proteins. Such E1A mimics are, for example, polypeptides which consist essentially of the amino acid residues of such an E1A region (e.g., the N-terminal region, CR1), DNA encoding the E1A region or small organic molecules which mimic the activity of the E1A region. The E1A region mimics are also the subject of the present invention, as are a method of enhancing apoptosis and sensitivity to chemotherapeutic agents and radiation in Rb deficient cells using the E1A mutants and/or mimics, a method of enhancing apoptosis and chemosensitivity and sensitivity to radiation in Rb deficient cells (e.g., tumor or malignant cells) in an individual who is being treated with chemotherapeutic agents and/or radiation using the E1A mutants and/or mimics, a method of identifying an E1A mutant and a method of identifying a molecule which mimics the function of an E1A mutant as described herein.

### BRIEF DESCRIPTION OF THE DRAWINGS

FIG. 1 is a schematic representation of E1A mutants, in which the following are represented: E1A: the 243-amino acid product of the 12S E1A. The two conserved regions that exhibit sequence homology among various adenovirus serotypes are indicated as CR1 and CR2 (white boxes). In the E1A mutants, deletions are indicated by gaps, and point mutations by an “x”. ΔN, ΔCR1 and ΔCR2 are deletions of amino acids 2 to 36, 68-85, and 120-140, respectively. The pm47/124 mutant has tyrosine to histidine changes at amino acids 47 and 124. Cellular proteins able to interact with each E1A mutant in co-immmunoprecipitations are indicated.

FIGS. 2A, 2B and 2C are graphic representations of results of assessment of the ability of two distinct regions of E1A to promote chemosensitivity in mouse fibroblasts (MEF) and human fibroblasts (IMR90). FIG. 2A shows results in primary human (IMR90) fibroblasts and FIGS. 2B and 2C show results in primary mouse fibroblasts. Primary mouse (MEF) or human (IMR90) fibroblasts were infected with an empty vector (vector, open circles), vectors expressing full-length E1A (E1A, closed circles), or the following mutants: ΔN (closed square), ΔCR1 (open square), ΔCR2 (open triangles), and pm47/pm124 (closed triangles). After selection in puromycin to remove uninfected cells, cells were plated in multiwell dishes and treated with the indicated concentrations of adriamycin (FIGS. 2A and 2B) or serum (FIG. 2C). Cell viability was determined by trypan blue exclusion at 24 or 48 hours for adriamycin treatment or serum withdrawal, respectively. Each point represents the mean ±SD from at least three separate experiments.

FIGS. 3A-3F are graphic representations of results which show that separate E1A functions cooperate to confer chemosensitivity. IMR90 or MEF cell populations expressing E1A (closed circles), ΔN (closed squares), ΔCR1 (open squares), ΔCR2 (closed triangles), ΔN and ΔCR2 (A and B, open circles), ΔN and ΔCR1 (C and D, open circles), or ΔCR1 and ΔCR2 (E and F, open circles) were generated by retroviral infection. Multiple E1A mutants were introduced sequentially, with selection for each mutant after each infection. Cell populations were treated with adriamycin and viability was determined 24 hours later by trypan blue exclusion. Each point represents the average ±SD of the data from at least three separate experiments.

FIGS. 4A-4H are graphic representations of results which show that inactivation of Rb by CR2 is required for chemosensitivity. Wild-type, Rb^{-/-}, p107^{-/-}, and p130^{-/-} MEFs expressing either E1A (closed circles), ΔCR2 (open circles, top panels), or ΔN (open circles, bottom panels) were generated by retroviral infection followed by brief selection with puromycin. After selection, cells were treated with adriamycin, and 24 hours later cellular viability was determined by trypan blue exclusion. Each point represents the mean ±SD from at least three separate experiments.

FIG. 5 is a graphic representation of results of assessment of chemosensitivity in the presence of pm47/124 in wild-type (Rb^{+/+}; open circles) and Rb^{-/-} (open triangles) cells and in the presence of E1A in wild-type (closed circle) and Rb^{-/-} (closed circle) cells, treated as described in the description of FIGS. 4A-4H.

FIGS. 6A-6C are graphic representations of results of chemosensitivity of Snos2 osteosarcoma cells in the presence of E1A mutants.

FIGS. 7A-7C are graphic representations of results of chemosensitivity of U2OS osteosarcoma cells in the presence of E1A mutants.

FIG. 8 is a graphic representation of results of chemosensitivity of BT-549 breast carcinoma cells in the presence of E1A mutants.

### DETAILED DESCRIPTION OF THE INVENTION

As described herein, two functionally distinct adenovirus E1A activities which act in concert to promote p53 accumulation and chemosensitivity in normal non-tumorigenic mammalian (e.g., mouse, rat, rabbit, dog, cat, monkey, human) cells have been identified. As also described, one of these functions is shown to involve inactivation of the Rb gene product. Identification of the E1A regions and demonstration that functionally distinct E1A regions cooperate to confer chemosensitivity and that E1A regions that promote chemosensitivity also induce p53 are described in detail in the Exemplification.

Work described herein was carried out to determine how E1A promotes sensitivity to toxic agents, such as chemotherapeutic agents or drugs and irradiation. To do so, a series of E1A mutants were introduced into primary human and murine fibroblasts; this permitted analysis of E1A in genetically-normal cells and outside the context of adenovirus infection. Mutations that disrupted E1A-induced apoptosis and chemosensitivity were separated into two complementation groups, which correlated precisely with their ability to associate with either the Rb-related proteins or p300/CBP proteins. E1A mutants incapable of binding Rb, p107 and p130 conferred chemosensitivity to fibroblasts derived from Rb-deficient mice, but not fibroblasts from mice lacking either p107 or p130. Thus, inactivation of Rb, but not p107 or p130, is required for chemosensitivity induced by E1A. E1A mutants which are defective in binding p300 and/or CBP and bind Rb are also described. They serve as the basis for designing or identifying molecules which mimic E1A regions and are expected to be useful for enhancing apoptosis and chemosensitivity in Rb protein deficient cells. As also shown by Applicant, the same regions of E1A that promote drug-induced apoptosis induce p53 as well. These data demonstrate that p53 accumulation and chemosensitivity are linked to E1A's oncogenic potential and provide a strategy to preferentially induce apoptosis in Rb-deficient tumor cells.

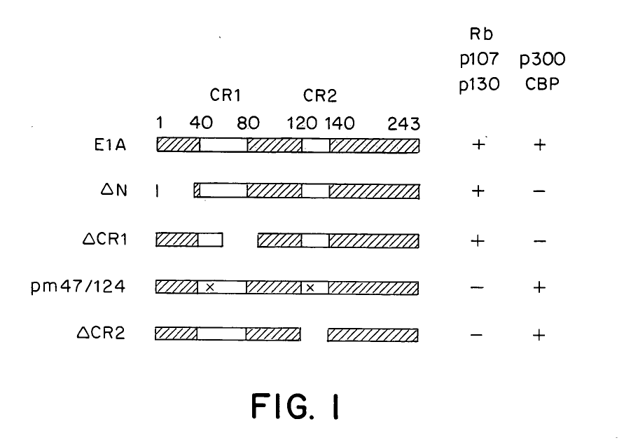

The domains of the E1A oncoprotein described herein are defined with reference to the E1A oncoprotein amino acid sequence. There are many adenovirus serotypes. E1A oncoproteins of all serotypes are encompassed by the term “E1A oncoprotein amino acid sequence” as used herein. The E1A gene expresses several alternatively spliced transcripts, including the 12S and 13S messages encoding 243 (243R) and 289 (289R) amino acid oncoproteins, respectively. Perricaudet, M., Nature, 281:694-696 (1979); Horwitz, M. S., “Adenoviridae and their Replication” In: Virology, Second Edition (B. N. Fields et al., ed.) Raven Press, Ltd., New York, pp. 1679-1695 (1990). The 289R protein contains three regions that are conserved between different adenovirus serotypes. These regions are designated conserved regions 1, 2 and 3 (CR1, CR2 and CR3). CR3 encodes a domain required for transcriptional activation of other viral genes and is absent in the 243 R protein; CR1 and CR2 are present in both E1A proteins and are essential for many E1A activities, including oncogenic transformation. E1A domains identified are represented schematically with reference to the E1A 12S oncoprotein in FIG. 1. An N-terminal domain is located approximately from amino acid residue 2 to amino acid residue 36. E1A domain CR2 is located from approximately amino acid residue 120 to approximately amino acid residue 140. E1A domain CR1 is located from approximately amino acid residue 40 to amino acid residue 80. Alteration of one or more of these domains in such a manner that the resulting E1A mutant is defective for (does not enhance) apoptosis and chemosensitivity in normal non-tumorigenic cells is also described, as are E1A mutants defective for apoptosis and chemosensitivity in normal non-tumorigenic mammalian cells which enhance apoptosis and sensitivity to toxic agents of Rb protein deficient cells.

E1A mutants which do not enhance apoptosis or chemosensitivity when present in normal non-tumorigenic mammalian cells, but enhance apoptosis and chemosensitivity in Rb protein deficient or mutant mammalian cells are also the subject of this invention. Rb protein deficient or mutant cells include cells which lack a Rb gene or contain a mutant Rb gene and, as a result, do not produce Rb or produce Rb at reduced levels and cells in which Rb is produced and is inactivated, directly or indirectly. Rb is inactivated directly, for example, when it is bound by an agent which inhibits its function or when it is degraded (e.g., by a cellular enzyme). For example, Rb can be inactivated through the effects or actions of agents, such as an infectious agent (e.g., papillomavirus) or a chemical which inactivates Rb. Many human cervical cancers, for example, are caused by papillomavirus infections. These tumors express papillomavirus E7, which acts like E1A (E1A CR2) to inactivate Rb. Rb is inactivated indirectly, for example, as a result of an alteration in the pathway by which Rb acts as a tumor suppressor. Normally, Rb protein is regulated by phosphorylation; when the protein is phosphorylated, it is off. If a change occurs in the cell such that Rb protein is permanently off (inactivated), the result is equivalent to what occurs when Rb protein is mutated. Certain enzymes, (the cyclin-dependent kinases (cdk)), phosphorylate Rb protein and turn it off. If a mutation produces too much cyclin, the cdk is always on and Rb protein is always off. Cyclin D is, in fact, an oncogene which is mutated in many tumor types. In addition, the p16 tumor suppressor acts to inhibit the cdk, indirectly preventing phosphorylation of Rb protein, with the result that Rb protein remains on. However, a very large number of tumors have inactivated p16; p16 is a tumor suppressor and, thus, the cdk cannot be turned off and Rb protein remains permanently off. Thus, mutations in p16 are, in essence, roughly equivalent to mutations in Rb. Mutations in the cdk catalytic subunit which prevent p16 from binding have also been described; cdk remains on and Rb protein remains off. Thus, the presence of papillomavirus E7, cyclin D overexpression, certain mutant cdk (e.g., a cdk 4 with an arg to cys alteration at position 24) and p16 mutations represent states equivalent to Rb protein mutation. Rb protein deficient or mutant cells also include, for example, cells derived from a retinoblastoma knockout mammal (e.g., mouse) and tumor cells (e.g., Snos2 osteosarcoma cells, U2OS osteosarcoma cells and BT-549 breast carcinoma cells). E1A mutants described herein can be used to enhance apoptosis and sensitivity to chemotherapeutic agents and radiation in cells in which these equivalent states occur.

Thus, E1A mutants of the present invention, such as E1A mutants in which an internal domain (e.g., CR2) is deleted or altered, are useful in Rb protein deficient tumor cells to increase chemosensitivity or sensitivity to radiation and apoptosis.

E1A mutants which differ from E1A 12S or E1A 13S by at least one amino acid residue and which do not enhance apoptosis or chemosensitivity in normal non-tumorigenic mammalian (e.g., mouse, dog, rat, rabbit, cat, monkey, human) cells, but do so in Rb protein deficient or mutant cells, are the subject of this invention. E1A mutants differ from E1A oncoprotein in that at least one amino acid residue is deleted, modified or replaced by an amino acid residue other than that present in the corresponding position in E1A. In one embodiment, one or more amino acid residues at or near the E1A N-terminus is (are) deleted, modified or replaced. In a specific embodiment, at least one amino acid of the N-terminal domain from about amino acid residue 2 to about amino acid residue 36 is deleted, modified or replaced. For example, in one type of E1A mutant, amino acid residues 2 to 36 are deleted, modified or replaced. A shorter domain (e.g., fewer than 5, 5 to 10, 5 to 15, 5 to 20, 5 to 25, 5 to 35) or a longer domain (e.g., more than 35 amino acid residues but fewer than 40 amino acid residues) can be deleted, modified or replaced. In a specific embodiment, E1A mutants lack amino acid residues 2 to 36 of E1A.

In a second embodiment, one or more amino acid residues of internal domain CR2 (approximately amino acid residues 120 to 140) are deleted, modified or replaced and the resulting E1A mutant has the desired effect on cytotoxicity. For example, in one type of mutant, amino acid residues 120 to 140 are deleted, modified or replaced. A shorter domain or portion of CR2 can also be deleted, modified or replaced (e.g., amino acid residues 120 to 135, 120 to 130, 125 to 140, 125 to 135, 125 to 130 can be deleted, modified or replaced). In a specific embodiment, the E1A mutant lacks amino acid residues 120 to 140. Another E1A mutant of the present invention includes deletion, modification or replacement of an amino acid residue in domain CR1 and deletion, modification or replacement of an amino acid residue in domain CR2. For example, the tyrosine residues at positions 47 and 124 are replaced with histidine residues or another suitable amino acid residue. In another E1A mutant, one or more amino acid residues of domain CR1 can be deleted, modified or replaced. Amino acid residues 68-85 or a shorter domain or portion of CR1 can be deleted, modified or replaced (e.g., amino acid residues 68 to 80, 68 to 75, 68 to 70, 70 to 85, 75 to 85). In one embodiment, the E1A mutant lacks amino acid residues 68 to 85.

Other E1A mutants can be made, with reference to the domains identified herein and through the use of known methods. E1A mutants produced in this manner can be further characterized by or defined with reference to their ability to bind or interact with Rb or p300/CBP proteins. For example, additional E1A mutants, such as mutants in which a portion of CR2 other than amino acid residue 120 to 140 is deleted, can be produced and assessed for their ability to bind Rb and/or p300/CBP.

As described herein, the effects of E1A mutants of the types described herein on apoptosis and chemosensitivity, as well as on p53 accumulation, have been assessed. As also shown herein, E1A mutants lacking amino acid residues 2 to 36 (referred to here as E1A ΔN mutants) are unable to enhance or promote chemosensitivity when they are expressed in normal cells, even when expression levels are comparable to levels of E1A expressed in the same cell type under the same conditions, and E1A mutants lacking amino acid residues 120 to 140 (referred to herein as E1A ΔCR2) are unable to enhance or promote chemosensitivity in normal cells. This is in sharp contrast to full-length E1A, which promotes apoptosis in normal, non-tumorigenic cells. As a result, E1A-expressing cells are more sensitive to the toxic effects of anti-cancer agents than cells in which E1A is not expressed. Cells that co-express both mutant proteins (E1A ΔN and E1A ΔCR2) behave like cells expressing full-length E1A and readily undergo apoptosis following treatment with anti-cancer agents. Thus, work described herein shows that two functionally distinct regions of E1A oncoprotein are essential for chemosensitization of normal cells.

It has also been determined that the E1A ΔCR2 mutant is defective for (does not enhance) apoptosis and chemosensitivity because it is unable to bind and inactivate retinoblastoma (Rb) protein. As described in the Exemplification, this was demonstrated by introducing E1A ΔCR2 into fibroblasts lacking p107 or p130. If amino acid residues 120 to 140 contribute to apoptosis and chemosensitivity by functionally inactivating Rb protein, then E1A ΔCR2 should be able to promote chemosensitivity in cells which lack the retinoblastoma gene (and, thus, already lack functional Rb protein). The E1A ΔCR2 mutant did not promote chemosensitivity in normal, p107-deficient cells, but enhanced the chemosensitivity of Rb protein deficient cells to the levels observed with full-length E1A. This supports the role of amino acid residues 120 to 140 in causing chemosensitivity by binding to and inactivating Rb protein. Therefore, mutant forms of E1A which are unable to bind Rb protein are defective in promoting chemosensitivity in normal cells, but markedly enhance apoptosis in cells which lack Rb protein.

As a result of work described herein, cells in which the retinoblastoma gene is inactivated, such as in individuals with familial retinoblastoma or one of a variety of sporadic human tumors, can be made more sensitive to toxic therapeutic agents, such as anti-cancer drugs or radiation. Thus, a method of specifically making tumor cells containing a mutant Rb protein more sensitive to toxic therapeutic agents (e.g., chemotherapeutic agents, such as cytotoxic anti-cancer drugs, and/or irradiation), while normal (nontumor) cells are unaffected or affected to a lesser extent, is a subject of the present invention, as are agents or drugs useful to specifically enhance Rb mutant tumor cell sensitivity to the chemotherapeutic agents and/or irradiation.

In one embodiment of the present method of enhancing Rb mutant tumor cell chemosensitivity, E1A mutants which fail to inactivate Rb protein are administered to an individual to whom toxic therapeutic agents, such as cytotoxic anti-cancer drug(s) and/or irradiation, are also administered to kill Rb mutant cells. For example, an E1A mutant which lacks an internal region, such as CR2 (amino acid residues 120 to 140), can be administered to the individual. Alternatively, a molecule or compound which mimics the function of the CR2 mutant can be administered. A CR2 “mimic”, like the E1A ΔCR2 mutant which lacks amino acid residues from about 120 to about 140 will not enhance chemosensitivity or apoptosis in normal cells, does not bind Rb protein and will enhance chemosensitivity and apoptosis in Rb protein deficient cells, such as tumor cells. As a result, Rb protein deficient cells are rendered more sensitive to chemotherapeutic agents. Alternatively, an E1A mutant which lacks a portion of the CR2 region can be administered. An E1A mutant in which a mutation, deletion or replacement of at least one amino acid residue in CR1 and a mutation, deletion or replacement of at least one amino acid residue in CR2 also enhances chemosensitivity in Rb mutant cells. It can be administered in an embodiment of the present method of enhancing sensitivity to chemotherapeutic agents and/or radiation. For example, E1A mutants in which the tyrosine residue at position 47 and at position 124 is each replaced with a histidine residue can be administered to enhance chemosensitivity of Rb^{-/-} cells.

In these embodiments, E1A mutants which enhance chemosensitivity or :sensitivity to radiation of Rb mutant cells are administered as E1A mutant protein or in a gene therapy protocol in which a nucleic acid construct encoding the appropriate E1A mutant is administered and expressed. More than one E1A mutant or more than one E1A mutant-encoding DNA or RNA construct can be administered to the individual.

The E1A mutant-encoding nucleic acid can be DNA or RNA. It can be administered in the form of an expression construct which includes additional sequences sufficient for expression of the E1A mutant in recipient cells. For example, a recombinant vector of viral, mammalian avian or bacterial origin (e.g., a replication defective recombinant viral vector such as a retroviral vector or adenoviral vector), can be used. Alternatively, it can be administered as “naked” DNA (or RNA) which relies on the expression machinery of recipient cells for its expression. In either case, the E1A mutant-encoding nucleic acid can be introduced into recipient cells, which are then introduced into an individual (where the E1A mutant is expressed) or can be introduced directly into an individual, in whom they enter cells and are expressed. In this embodiment, chemosensitivity of tumor cells lacking Rb protein is enhanced, but non-malignant cells (which have normal Rb protein) are not affected (their chemosensitivity is not enhanced). Anti-cancer agents which can be used in this embodiment include all cytotoxic anti-cancer drugs, such as, but not limited to, adriamycin (ADR), vincristine, vinblastine, 5-fluorouracil, cisplatin, tumor necrosis factor (TNF) and etoposide, and γ radiation. The amount of E1A mutant administered is determined empirically, with reference to the cytotoxic anti-cancer drug used, the type and severity of the cancer being treated and patient characteristics (e.g., age, size, gender).

In a second embodiment, a small molecule which mimics the function of the E1A N-terminus is used and synergizes with a cytotoxic anti-cancer drug to specifically kill tumor cells containing mutant Rb protein. The N-terminus of E1A has been shown to be unable to enhance chemosensitivity, and, thus, such small molecules are not expected to be toxic to non-malignant cells, which contain normal Rb genes. The small molecule can be the E1A N-terminal domain. An N-terminal domain polypeptide or DNA or RNA encoding the N-terminal polypeptide can be introduced into an individual, as described above with reference to E1A mutants lacking conserved region 2. Alternatively, small molecules, such as small organic molecules, can be used. For example, molecules which mimic the effect of E1A on p300 or CBP can be used.

An E1A mutant which does not bind Rb protein (e.g., ΔCR2 or pm47(124)) or a mimic of such an E1A mutant can be administered to an individual in conjunction with an agent which, consists essentially of the amino acid residues of the E1A region deleted from a mutant, such as ΔN or CR1, and/or which does not associate with the p300/CBP proteins. The resulting agent (e.g., the E1A N-terminus or CR1) retains the ability to bind the p300/CBP proteins. Alternatively, an agent, other than a peptide, which mimics the effect of the peptide on p300 and/or CBP proteins can be administered with an E1A mutant which does not bind Rb protein.

E1A mutants and DNA or RNA encoding E1A mutants are also the subject of the present invention. E1A mutants include all mutants which fail to inactivate Rb protein in normal cells and promote sensitivity of Rb mutant cells to anti-cancer drugs and/or irradiation. These mutants differ from E1A protein by at least one amino acid residue. In one embodiment, E1A mutants lack conserved region 2 or a portion of conserved region 2. One example is E1A ΔCR2, which lacks amino acid residues 120 to 140. Additional examples of such E1A mutants include those in which a portion of CR2 is deleted (e.g., amino acid residues 120 to 135, 120 to 130, 125 to 140, 125 to 135, 125 to 130). In another embodiment, E1A mutants have a deletion, modification or replacement of at least one amino acid residue in CR1 and at least one amino acid residue in CR2. For example, an E1A mutant of this type is one in which the tyrosine residues normally present at positions 47 and 124 are replaced by histidine residues. Other amino acid residues can be deleted, modified or replaced, using known methods, to produce additional E1A mutants of this type. Other amino acid residue deletions which result in an E1A mutant which does not inactivate Rb can be made and assessed, as described herein, to determine whether they inactivate Rb.

Also the subject of this invention are molecules which mimic the function of the E1A N-terminus (e.g., an E1A mutant, which lacks the E1A N-terminus, binds Rb protein but does not bind p300/CBP). A polypeptide having the amino acid sequence of the E1A N-terminus or a molecule or compound which mimics the effect of the N-terminus on the relevant cellular target (e.g., p300 or CBP) is also a subject of this invention. Additional amino acid residues, as needed, can be incorporated into the polypeptide.

Similarly, a molecule or compound which mimics the effect of E1A CR1 is a subject of this invention. An E1A mutant which lacks at least a portion of CR1 has been shown to bind Rb protein but not to bind p300/CBP proteins. A polypeptide having the amino acid sequence of CR1 or a portion thereof (e.g., approximately amino acid residues 68 to 85) or a molecule or compound which mimics the effect of CR1 or a CR1 portion on p300 or CBP is a subject of this invention. Additional amino acid residues, as needed, can be incorporated into the polypeptide.

A method of identifying E1A mutants which do not inactivate Rb in normal cells and enhance sensitivity of Rb mutant cells to anti-cancer agents is also a subject of this invention. In the method, a vector (e.g., a retroviral vector) which comprises DNA (or RNA) encoding an E1A mutant to be assessed and sufficient additional components to result in expression of the E1A mutant-encoding DNA or RNA in a mammalian cell is introduced into wild-type (Rb^{+/+}) and Rb deficient (Rb^{-/-}) mammalian cells. Full-length E1A is expressed in wild-type cells as a control. The mammalian cells are maintained under conditions appropriate for expression of the E1A mutant or full-length E1A and are contacted with an agent which stimulates apoptosis (e.g., adriamycin, cisplatin, 5-fluorouracil, γ-radiation). Viability of the cells is assessed, for example as described in the Exemplification. For a particular mutant, if wild-type cells remain relatively insensitive to the agent which stimulates apoptosis and Rb deficient cells exhibit apoptosis to the same or a similar extent as wild-type cells expressing full-length E1A and stimulated by the same agent, the mutant is identified as an E1A mutant which does not inactivate Rb or enhance apoptosis in wild-type (Rb^{+/+}) cells and which does enhance apoptosis and chemosensitivity in Rb deficient or mutant mammalian cells. E1A mutants identified in this manner are also the subject of this invention.

A method of identifying molecules or compounds which mimic the activity of an E1A mutant, as defined herein, such as an E1A mutant lacking CR2 or an E1A mutant having point mutations in CR1 and CR2 (e.g., pm47(124)) or the E1A N-terminus, is also the subject of this invention. ΔCR2 and pm47/124 bind p300/CRB. It is likely that the E1A N-terminus binds and inactivates the p300 protein, CBP or both. Therefore, it is likely that a small molecule that inactivates one or more of these proteins will synergize with Rb mutations to promote apoptosis in the absence of E1A protein. Identification or design of small molecules which mimic one of these mutants or the E1A N-terminus (or another E1A domain which interacts with Rb protein, p300 protein and CBP in the same manner as the N-terminus) can be carried out by screening candidate molecules for their ability to inhibit p300 protein and/or CBP. Molecules identified in this way can be subjected to a second screening step, in which their ability to enhance chemosensitivity (facilitate drug or radiation induced apoptosis) is assayed in Rb protein deficient cells (e.g., in Rb protein deficient mouse embryo fibroblasts, as described herein, in a similar system).

In one embodiment, the present invention relates to a method of identifying a molecule which mimics the function of an E1A mutant wherein the molecule does not bind and inactivate retinoblastoma protein in wild-type mammalian cells and which enhances sensitivity to chemotherapeutic agents and irradiation in retinoblastoma deficient mammalian cells. In the method, retinoblastoma deficient mammalian cells (Rb^{-/-} cells) and, as a control, wild type mammalian cells (Rb^{+/+} cells), are contacted with a molecule to be assessed and a sublethal dose of an agent which stimulates apoptosis in mammalian cells. As defined herein, a “sublethal dose” of an apoptotic agent is a dose such that cell death due to apoptosis does not occur or occurs to a lesser extent than if a lethal dose of the apoptotic agent were present. Whether apoptosis occurs in the wild-type mammalian cells and in the retinoblastoma deficient mammalian cells is determined and the extent to which apoptosis occurs in the retinoblastoma deficient mammalian cells to the extent to which apoptosis occurs in wild-type mammalian cells is then compared. Cell viability can be measured, for example, 24-48 hours later for differential toxicity in Rb^{+/+} and Rb^{-/-} cells. Viability of the cells is assessed, for example, using crystal violet, an MTT assay or methods described in the Exemplification. If apoptosis does not occur in the wild-type mammalian cells or occurs to a lesser extent than in the retinoblastoma deficient mammalian cells, then the molecule to be assessed is a molecule which mimics the function of an E1A mutant wherein the molecule does not bind and inactivate retinoblastoma protein in wild-type mammalian cells and enhances sensitivity to chemotherapeutic agents and irradiation in retinoblastoma deficient mammalian cells. For example, in the method, an E1A mimic (e.g., a ΔCR2 mimic) would effectively synergize with ADR to kill Rb −/− cells but produce little toxicity Rb +/+ cells. Molecules which mimic the function of an E1A mutant that are identified in this manner are also the subject of this invention.

The present invention is illustrated by the following examples, which are not intended to be limiting in any way.

Exemplification:

### EXAMPLE 1

Identification of E1A Regions Essential for E1A Ability to Promote Chemosensitivity and Induce p53

The following materials and methods were used to identify E1A regions essential for the ability of E1A to promote chemosensitivity and induce p53. Cells and Cell Culture. Mouse embryonic fibroblasts (MEFs) and normal diploid human lung fibroblasts (IMR90) were maintained in DMEM media supplemented with 10% fetal bovine serum and 1% penicillin-G/streptomycin sulfate (Sigma). MEFs were isolated as previously described (Serrano, M. et al., Cell 88:593-602 (1997)). Rb^{-/-} MEFs were obtained from T. Jacks (Jacks, T. et al., Nature 359:295-300 (1992)), p107^{-/-} and p130^{-/-} MEFs were from N. Dyson (Cobrinik, D. et al., Genes Dev. 10:1633-1644 (1996); Lee, M. H. et al., Genes Dev. 10:1621-1632 (1996)). The IMR90 cells overexpressed the murine ectopic receptor (Serrano et al., Cell 88:593-602 (1997)), allowing subsequent infection with ecotropic retroviruses. Gene transfer into MEFs was performed between passage three and six. Gene transfer into IMR90s were used between 30-30 populations doubling levels.

E1A mutants, retroviral vectors, and infections. The 12S E1A cDNA and 12S E1A deletion or point mutants (Kannabiran, C. et al., J. Virol. 67:507-515 (1993); Wang, H. G. et al., J. Virol. 67:476-488 (1993)) were a gift of M. Mathews and were subcloned into pLPC (Serrano, M. et al., Cell 88:593-602 (1997)) or pWZLHygro (J. P. Morgenstern, M. J. Zoller, J. S. Brugge, Ariad Pharmaceuticals). pLPC-12S co-expresses an E1A 12S cDNA with puromycin phosphotransferase (puro) and pWZL-12S co-expresses E1A with hygromycin phosphotransferase (hygro). The E1A mutant constructs used in this study were as follows: pLPC 12S.ΔN, pLPC 12S.ΔCR1, pLPC 12S.ΔCR2, pLPC 12S.pm47/124, pWZL 12S.ΔN, pWZL 12S.ΔCR1, and pWZL 12S.ΔCR2.

Ecotropic retroviruses were produced using the Phoenix packaging line (provided by G. Nolan, Stanford University) according to a previously described procedure (Pear, W. S. et al., Proc. Natl. Acad. Sci., USA 90:8392-8396 (1993); Serrano, M. et al., Cell 88:593-602 (1997)). Briefly, Phoenix cells were transfected with a pro-retroviral plasmid by the calcium phosphate co-precipitation method. After 24 to 36 hours the virus-containing media was placed on the appropriate target cells. Virus produced from 5×10⁶ Phoenix cells was used to infect 7.5×10⁵ target cells. Twelve hours after the infection, cells were placed into media containing 2.5 μg/ml puromycin (Sigma) or 100 μg/ml Hygromycin B (Boehringer Mannheim) to eliminate uninfected cells. When two separate E1A mutants were expressed together, they were introduced sequentially using separate markers after each round of infection. Infection efficiencies were greater than 50% prior to selection. More than 95% of the cells in the infected populations expressed E1A as determined by immunofluorescence. Each E1A mutant localized to the nucleus.

Cell viability and apoptosis. 1×10⁵ cells were plated into 12 well plates 24 hours prior to treatment. Twenty-four hours following treatment with adriamycin, or 48 hours after serum withdrawal, adherent and nonadherent cells were pooled and analyzed for viability by trypan blue exclusion. At least 200 cells were counted for each point. Null mutant fibroblasts were compared to cells derived from wild-type littermate controls.

Protein expression. Proteins were extracted in NP-40 lysis buffer (150 mM NaCl, 1% NP-40, 50 mM Tris, 1 mM PMSF, 1 mM EDTA, 2 μg/ml CLAP (chymostatin, leupeptin, antipain, and pepstatin) for one hour on ice with frequent vortexing. Lysates were normalized by Bradford method (BioRad), and 20 μg (for p53) or 10 μg (for E1A) of total protein was loaded in each lane. After electrophoresis, proteins were transferred to Immobilon-P membranes using “wet” transfer in transfer buffer (25 mM Tris, 192 mM glycine, 20% methanol w/v) for 1 hour at 100 volts. For detection of E1A, blots were probed using the M58(1:100) dilution) mouse monoclonal antibody, which recognizes an epitope retained in all E1A mutants studied. For p53, blots were probed using the rabbit polyclonal antibodies, CM1 (for human) or CM5 (for murine) (1:1000 dilution) (Company name). Proteins were visualized by ECL (Amersham), and equal sample loading was confirmed by India Ink staining of the membrane.

Results

A structure-function analysis was carried out to determine the regions of E1A required for apoptosis and chemosensitivity. A series of recombinant retrovirus vectors co-expressing various E1A mutants (FIG. 1) with either puromycin or hygromycin phosphotransferase were constructed. Earlier studies demonstrated that the 243 amino acid protein encoded by the E1A 12S cDNA was sufficient for apoptosis and chemosensitivity (Lowe, S. W. and Ruley, H. E., Genes Dev. 7:535-545 (1993); McCurrach, M. E., et al., Proc. Natl. Acad. Sci., USA 94:2345-2349 (1997)); hence, all mutants were derived from an E1A 12S cDNA (Kannabiran, C. et al., J. Virol. 67:507-515 (1993); Wang, H. G. et al., J. Virol. 67:476-488 (1993)). Each mutant is compromised in its ability to physically associate with either the p300/CBP (ΔN or ΔCR1) or pRB/p107/p130 (pm47/124 or ΔCR2) family of cellular proteins (FIG. 1) (Wang H. G. et al., J. Virol. 67:476-488 (1993)).

High-titer ecotropic retroviruses were generated using a transient retrovirus packaging system (Pear, W. S. et al., Proc. Natl. Acad. Sci., USA 90:8392-8396 (1993)). Virus supernatants were used to infect either normal diploid IMR90 human lung fibroblasts or primary mouse embryonic fibroblasts (MEFs), and pure populations of E1A-expressing cells were isolated by brief selection in the presence of puromycin or hygromycin B. All E1A mutant proteins were efficiently expressed. As a result, E1A was stably expressed in primary cell populations in the absence of additional adenoviral proteins (in a genetically normal background).

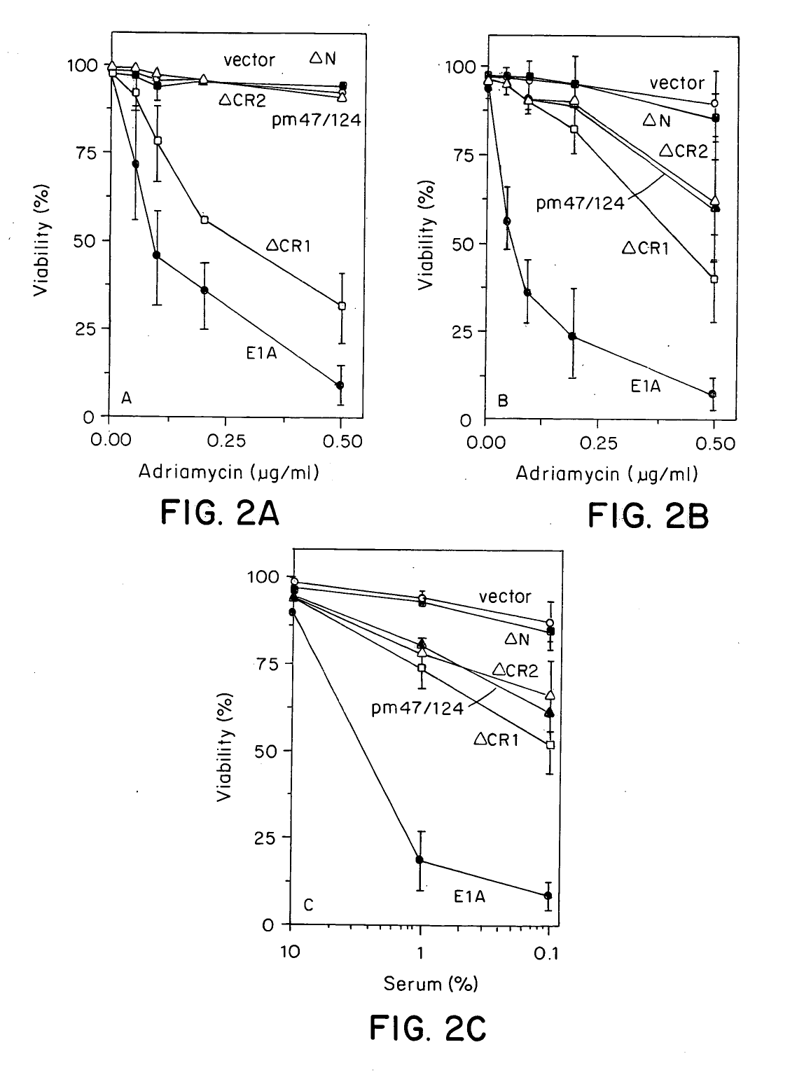

Results showed that multiple E1A regions are required for apoptosis and chemosensitivity in wild-type cells. Full-length E1A sensitized both human and mouse fibroblasts to the induction of apoptosis by a variety of agents (see, for example, FIGS. 2A-2C). As expected, mouse cells expressing E1A lost viability in a dose-dependent manner following adriamycin treatment or serum withdrawal (FIGS. 2B and 2C). Under these conditions, cell death is largely p53-dependent, since p53-deficient MEFs expressing E1A remained viable. Human cells also lost viability following adriamycin treatment, but not after serum withdrawal (FIG. 2A). In both cell types, the dying cells displayed features of apoptosis. (Lowe, S. W. et al., Cell 74:957-967 (1993); McCurrach, M. E., et al., Proc. Natl. Acad. Sci., USA 94:2345-2349 (1997)). Like uninfected cells, fibroblasts infected with an empty vector did not undergo apoptosis after either treatment (FIGS. 2A-2C). Thus, the retroviral vector itself had no effect.

All of the E1A mutants tested were defective in promoting apoptosis and chemosensitivity in both human and mouse fibroblasts (FIGS. 2A-2C). IMR90 cells expressing the ΔN, pm24/147, and ΔCR2 mutants were completely insensitive to adriamycin treatment (FIGS. 2A, 2B). Although IMR90 cells expressing the ΔCR1 mutant lost viability in a dose-dependent manner, cell death was substantially reduced compared to full-length E1A (35% vs 11% viable at 0.5 μg/ml, respectively) (FIG. 2A). Like IMR90s cells, MEFs expressing the ΔN and ΔCR1 mutant remained completely or partially insensitive to adriamycin treatment, respectively (FIG. 2B). MEFs expressing each E1A mutant were also defective in apoptosis following serum withdrawal, a treatment not known to produce cellular damage (FIG. 2C). The behavior of each E1A mutant was independent of the apoptotic stimulus, since similar patterns of chemosensitivity were observed following treatment of human and mouse cells with adriamycin, etoposide, cisplatin, 5-fluorouracil, or γ-radiation. These results showed that multiple regions of E1A are required for apoptosis following treatment with diverse agents.

Results presented herein also showed that functionally distinct regions of E1A cooperate to confer chemosensitivity. Each E1A mutant defective in apoptosis was also impaired for binding either the p300/CBP or Rb-related proteins (see FIG. 1), raising the possibility that these processes are related. To establish how many E1A functions contribute to apoptosis, combinations of E1A mutants were expressed in a trans complementation assay to determine whether the two mutations affected the same or separate functions. If two E1A mutants were defective because they lacked the same function, they would be unable to function in trans to confer chemosensitivity. Conversely, if two mutants were defective owing to loss of separate functions, then co-expressing these mutants should restore chemosensitivity. Therefore, E1A mutants were introduced sequentially into IMR90s and MEFs by retrovirus-mediated gene transfer using different selectable markers, the first producing puromycin resistance and the second resistance to hygromycin B. After the second round of selection, cell populations expressing each individual mutant, or the various mutant combinations, were treated with apoptosis-inducing stimuli and assayed for viability.

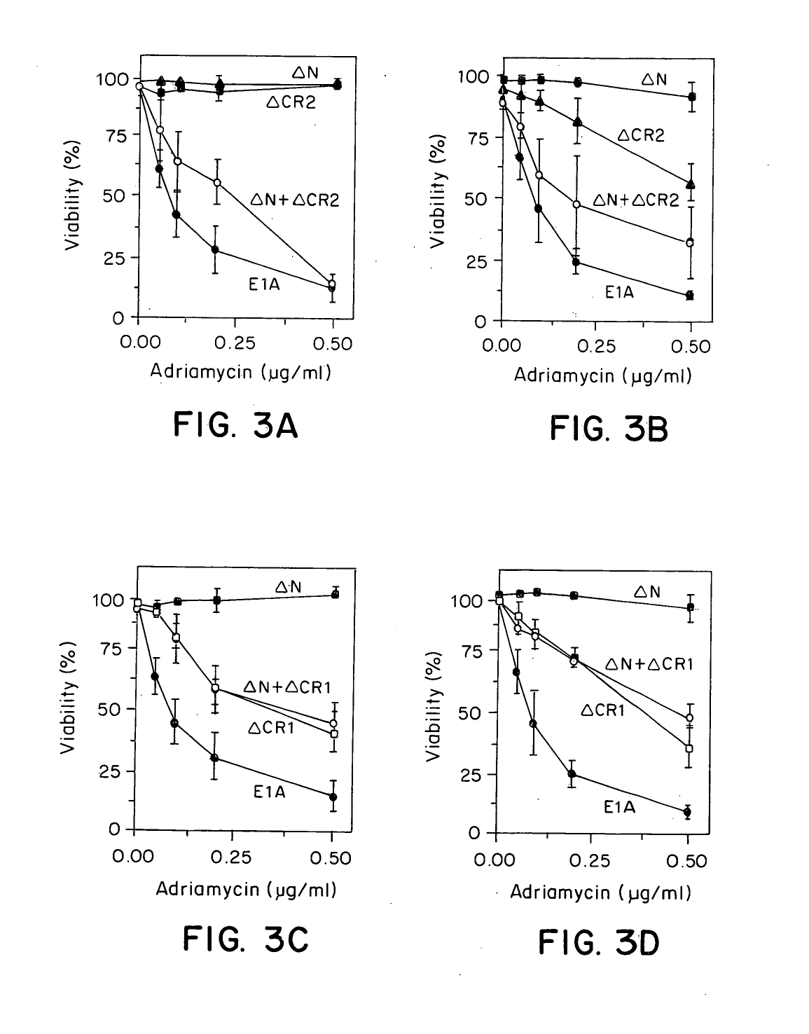

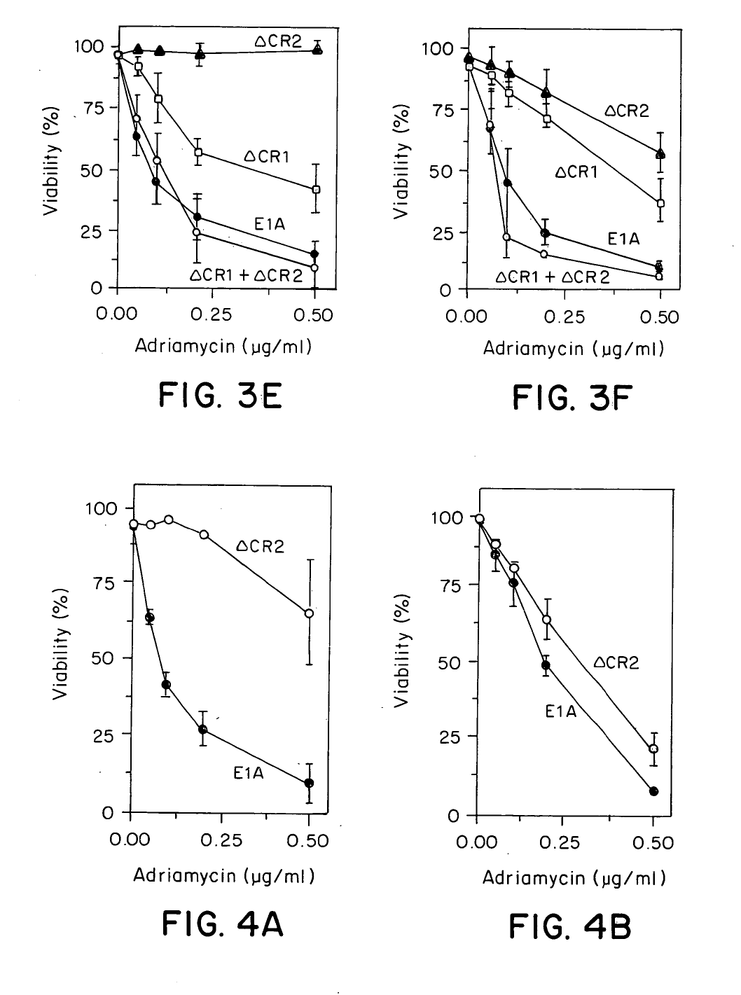

In both human and mouse fibroblasts, E1A mutants that bound different classes of cellular proteins were able to cooperate in trans to restore chemosensitivity, whereas those that bound the same class were not (FIG. 3). For example, although cells expressing either the ΔN mutant or the ΔCR2 mutant alone were insensitive to adriamycin-induced apoptosis, the levels of apoptosis in cells co-expressing these mutants approached those observed in cells expressing full-length E1A (FIGS. 3A and 3B). Similar results were observed when cells were treated with other anti-cancer agents or following serum withdrawal. Cells co-expressing the ΔCR1 and ΔCR2 mutants were as sensitive to adriamycin-induced apoptosis as cells expressing full-length E1A, again supporting the idea that each mutation affected a separate E1A function (FIGS. 3E, 3F). No increase in chemosensitivity was observed when cells were infected sequentially with the same E1A mutant (e.g., ΔN or ΔCR1) when compared to cells infected only once. This indicates that the observed cooperativity between ΔN and ΔCR1 with ΔCR2 did not result from increased gene dosage, but rather, was due to synergy between separate E1A functions. These results demonstrate that multiple E1A activities contribute to chemosensitivity.

In contrast, the ΔN and ΔCR1 mutants failed to restore chemosensitivity when expressed in trans: cells co-expressing the ΔN and ΔCR1 E1A mutants behaved identically to cells expressing the partially defective ΔCR1 mutant alone (FIGS. 3C and 3D). As discussed above, both ΔN and ΔCR1 restored chemosensitivity when co-expressed with ΔCR2, implying that the ΔN and ΔCR1 mutations did not produce global aberrations in E1A structure, but rather, disrupted the same discrete function. Of note, both ΔN and ΔCR1 are also defective in binding the p300/CBP proteins (see FIG. 1). The fact that two E1A mutants that fail to bind p300/CBP are defective for apoptosis because they affect the same function strongly implies that binding of one or more of these proteins is required for chemosensitivity.

The role of CR2 in chemosensitivity was also assessed. Conserved region 2 (CR2) is required for the physical association between E1A and the Rb-related proteins. In principle, CR2 could contribute to chemosensitivity by inactivating one or more of these proteins or by affecting some other cellular activity. If CR2 promotes chemosensitivity by inactivating a single Rb-related protein, then the ΔCR2 mutant should behave like full-length E1A in cells lacking this crucial target. Since all of the Rb-related genes have been disrupted in mice (Cobrinik, D. et al., Genes Dev. 10:1633-1644 (1996); Jacks, T. et al., Nature 359:295-300 (1992); Lee, M. H. et al., Genes Dev. 10:1621-1632 (1996)), this hypothesis could be tested definitively.

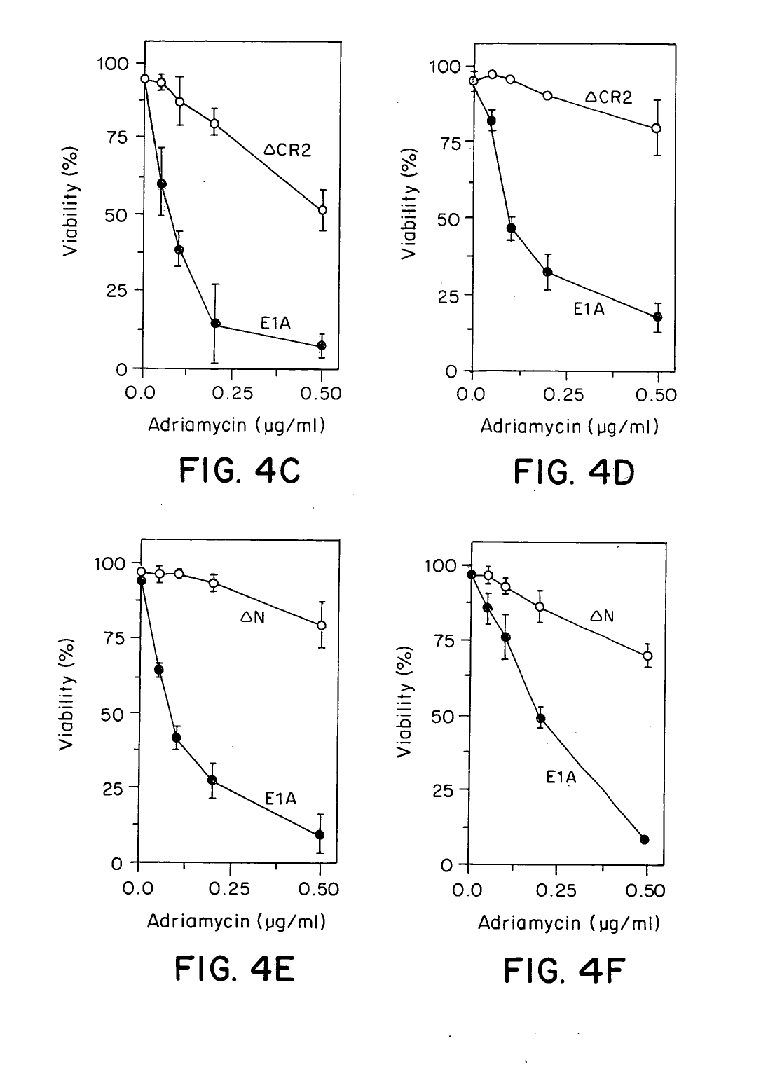

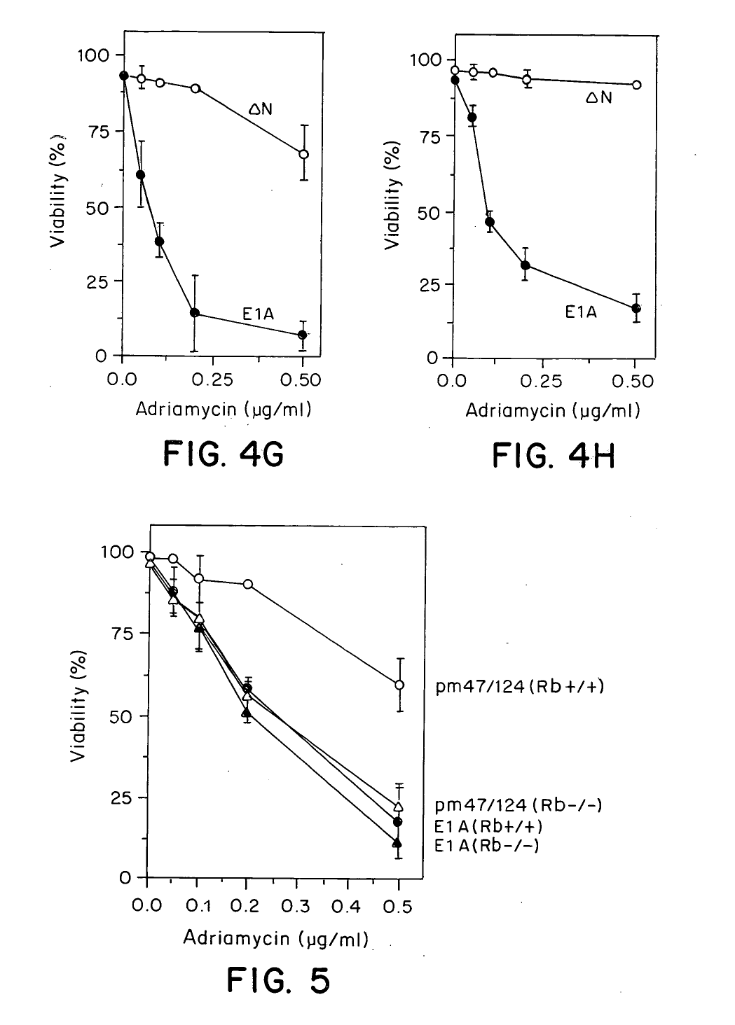

E1A and the ΔCR2 mutant were introduced into wild-type, Rb^{-/-}, p107^{-/-}, or p130^{-/-} MEFs as before, and the resulting populations were treated with apoptosis-inducing stimuli (FIGS. 4A-4H). Adriamycin treatment induced similar levels of apoptosis in cells expressing full-length E1A, irrespective of their genotype. Thus, as expected, loss of the Rb-related proteins does not impair apoptosis. Furthermore, MEFs infected with the empty vector were insensitive to adriamycin treatment, demonstrating that loss of either pRb, p107, or p130 was not sufficient to produce chemosensitivity.

Concordant with previous results, wild-type MEFs expressing the ΔCR2 mutant are relatively insensitive to adriamycin treatment, indicating that the ΔCR2 is defective at promoting chemosensitivity (FIGS. 4A-4H). Likewise, p107^{-/-} and p130^{-/-} cells expressing ΔCR2 remained insensitive to adriamycin treatment (FIGS. 4E and 4D). By contrast, Rb^{-/-} cells expressing ΔCR2 or pm47/124 were as sensitive to adriamycin-induced apoptosis as cells expressing full-length E1A (FIG. 4B and FIG. 5). This synergy was specific for ΔCR2 and pm47/124, since the ΔN mutant remained defective in all cell types (FIGS. 4E-4H). Thus, inactivation of pR—but not p107 or p130—is the critical function of CR2 important for apoptosis. Furthermore, E1A mutants unable to bind Rb are defective in normal cells but promote apoptosis in cells with mutant Rb genes.

Further work demonstrated that regions of E1A that promote chemosensitivity also induce p53. Cells expressing E1A accumulate p53 protein due, in part, to increased p53 stability (Lowe, S. W. and Ruley, H. E., Genes Dev. 7:535-545 (1993)). To determine whether the regions of E1A required for p53 accumulation co-localize with those required for chemosensitivity, we examined the ability of each E1A mutant to induce p53. Cells expressing full-length E1A displayed an increase in steady state p53 protein levels. The ΔN and ΔCR2 mutants produced only a slight increase in p53 levels when expressed in IMR90 cells, and no increase when expressed in MEFs. However, p53 approached wild-type levels when the ΔN and ΔCR2 mutant were expressed in trans. Furthermore, the ΔCR2 mutant induced p53 in Rb-deficient cells, greater than levels induced by full length E1A in wild type cells. Conversely, the ΔCR2 mutant failed to elevate p53 levels in p107 and p130-deficient cells. Rb-deficient cells infected with the empty vector displayed no increase in p53 levels. Thus, the same functions of E1A required for apoptosis and chemosensitivity also induce p53.

Discussion

All E1A mutants tested showed marked reduction in apoptosis potential in both primary human and mouse fibroblasts, and the requirement for each E1A region was independent of the apoptotic stimulus. These regions correlated precisely with the ability of E1A to associate with the p300/CBP and the Rb-related proteins. Co-expression of E1A mutants binding separate classes of cellular proteins functioned in trans to confer chemosensitivity, whereas expression of mutants binding the same cellular proteins did not. Thus, this study genetically defines at least two E1A functions that act in concert to promote apoptosis and chemosensitivity.

The results described herein provide strong genetic evidence that E1A's interaction with the p300/CBP proteins is critical for chemosensitivity. Specifically, a genetic complementation test was used to demonstrate that two spatially separate E1A mutations, both known to disrupt p300/CBP binding (ΔN and ΔCR1), affect the same E1A function involved in chemosensitivity. Whereas ΔCR1 is unable to associate with p300/CBP in immunoprecipitations, it retains some capacity to affect p300/CBP functions in cells (Kannabiran, C. et al., J. Virol. 67:507-515 (1993); Lee, J. S. et al., Genes Dev. 9:1188-1198 (1995)). By contrast, the ΔN mutant is completely defective in p300/CBP interaction using both immunoprecipitations and functional assays. Perhaps this explains why the ΔN and ΔCR1 mutants displayed a complete and partial defect in apoptosis, respectively (see FIGS. 3A-3F). p300 and CBP are both transcriptional co-activators and histone acetyltransferases (Cell 87:953-959; reviewed in Nature 383:22-23), and E1A binding to p300 produces global changes in transcription. Further functional analysis will undoubtedly provide insight into the role of p300 and CBP in apoptosis.

In addition to the p300/CBP binding domain, a second E1A function is required for apoptosis and chemosensitivity. Using primary fibroblasts derived from Rb^{-/-}, p107^{-/-}, or p130^{-/-} deficient mice, it has been conclusively demonstrated that this involves inactivation of Rb, but not p107 or p130. Interestingly, inactivating mutations in the retinoblastoma gene occur in a variety of human cancers; by contrast, mutations in p107 or p130 have not been observed (Weinberg, R. A., Cell 81:323-330 (1995)). The fact that E1A promotes chemosensitivity by inactivating a tumor suppressor underscores the utility of viral oncogenes to identify processes relevant to human cancer. Furthermore, the critical role of Rb inactivation in apoptosis reiterates the fundamental relationship between tumorigenesis and chemosensitivity.

p53 protein accumulates in cells expressing E1A, and this increase correlates with the involvement of p53 in apoptosis (Lowe, S. W. et al., Proc. Natl. Acad. Sci., USA 91:2026-2030 (1994); Lowe, S. W. and Ruley, H. E., Genes Dev. 7:535-545 (1993)). As demonstrated herein, the same functions of E1A that promote apoptosis and chemosensitivity also induce p53. These regions are also required for E1A's transforming activities (Whyte, P. et al., Nature 334:124-129 (1988), implying that p53 accumulation, chemosensitivity, and oncogenic potential arise from the same E1A activities (see also, Querido, E. et al., J. Virol. 71:3526-3533 (1997)). This suggests that p53 accumulation is a cellular response to oncogenic “stress” rather than a direct effect of E1A on p53. Interestingly, extracts from E1A-expressing cells possess a discrete factor that reproduces some of the pro-apoptotic activities of E1A in cell-free systems (Fearnhead, H. O. et al., Genes Dev. 11: 1266-1276 (1997)). The nature of this factor may shed light on the links between p53, chemosensitivity and cell-cycle control.

The retinoblastoma gene is mutated in a wide variety of human cancers, and the Rb pathway is inactivated in the vast majority of cancer cells. The work described herein provides a strategy to specifically kill cancer cells with defective Rb function. In normal cells, at least two processes affected by E1A are necessary to promote chemosensitivity—Rb inactivation and apparently disruption of some p300/CBP function. The Rb-inactivating function of E1A is dispensable for chemosensitivity in Rb-deficient cells. Consequently, such E1A mutants, or small molecules which mimic their action, should synergize with standard chemotherapeutic agents to specifically induce apoptosis Rb mutant tumor cells. Although p53 potentiates apoptosis under the conditions used in this study, E1A can promote chemosensitivity in p53-deficient cells (Lowe, S. W. et al., Cell 74:957-967 (1993); McCurrach, M. E., et al., Proc. Natl. Acad. Sci., USA 94:2345-2349 (1997)). Consequently, this therapeutic approach may not strictly depend on the presence of wild-type p53.

### EXAMPLE 2

Characterization of E1A Mutant E1A ΔCR2

The E1A mutant E1A ΔCR2 was introduced into primary mouse fibroblasts lacking pRb, p107 or p130 (derived from gene knockout mice). If CR2 contributes to apoptosis and chemosensitivity by inactivating Rb, then the otherwise defective E1A ΔCR2 mutant should be fully able to promote chemosensitivity in cells which lack the Rb gene and, hence, have no functional Rb protein. Results of assessment of the E1A mutant to promote chemosensitivity are shown in FIGS. 3A-3F. The E1A ΔCR2 mutant did not promote chemosensitivity in normal, p107-deficient and p130-deficient cells, but did enhance the chemosensitivity of Rb-deficient cells to the levels observed with full-length E1A (FIGS. 3D-3F, open triangles). This indicates that amino acid residues 120-140 contribute to chemosensitivity by inactivating Rb protein (pRb), but not p107 or p130. As a result, mutant forms which are unable to bind Rb protein are defective in promoting chemosensitivity in normal cells, but markedly enhance apoptosis in cells lacking Rb. FIGS. 3A-3C are controls, showing that loss of Rb, p107 or p130 enhanced the chemosensitivity of an N-terminal mutant.

### EXAMPLE 3

E1A Mutant Defective in Binding Rb Enhance the Chemosensitivity of Human Tumor Cell Lines with Known Defects in the Rb Pathway

A control vector, E1A or the E1A mutants (ΔN or ΔCR2) were introduced into tumor cells via retroviral mediated gene transfer and analyzed for drug cytoxicity within a few days of gene transfer. Cell populations were treated with adriamycin or tumor necrosis factor a (or both). It has been previously shown that adriamycin is more effective at inducing apoptosis in cells with normal p53 function (Lowe, S. W., et al., Cell, 74:954-967 (1993); by contrast, tumor necrosis factor induces apoptosis independently of p53 (Lanni, J. S., et al., Proc. Natl. Acad. Sci. USA, 94:9679-9683 (1997). These drugs were selected since ADR and TNF synergize to promote apoptosis in E1A expressing cells.

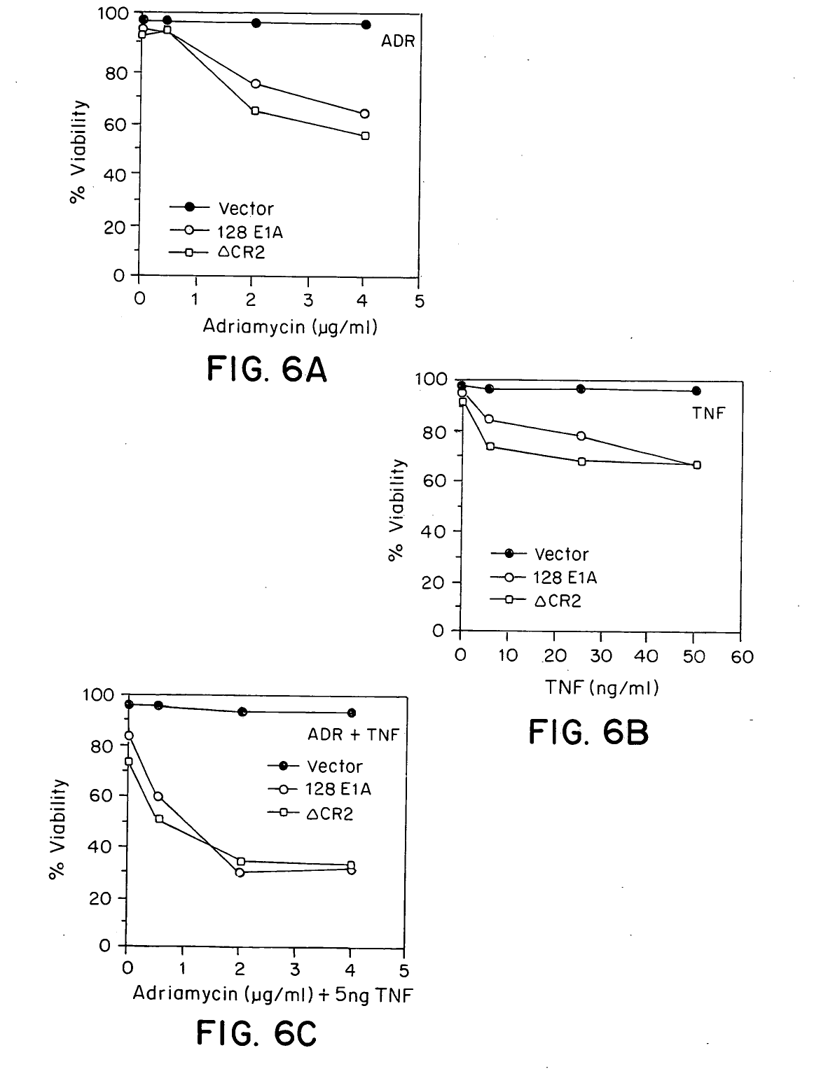

Snos2 osteosarcoma cells contain Rb and p53 mutations. These cells do not undergo apoptosis following treatment with either adriamycin or TNF at the doses tested (closed circles). The results, which are shown in FIGS. 6A-6C, demonstrate that E1A (open circles) enhances the cytotoxicity of both ADR and TNF (or the combination), and, importantly, E1A ΔCR2 (open squares), an E1A mutant which is unable to enhance drug cytotoxicity in normal cells, promotes chemosensitivity as well as full length E1A.

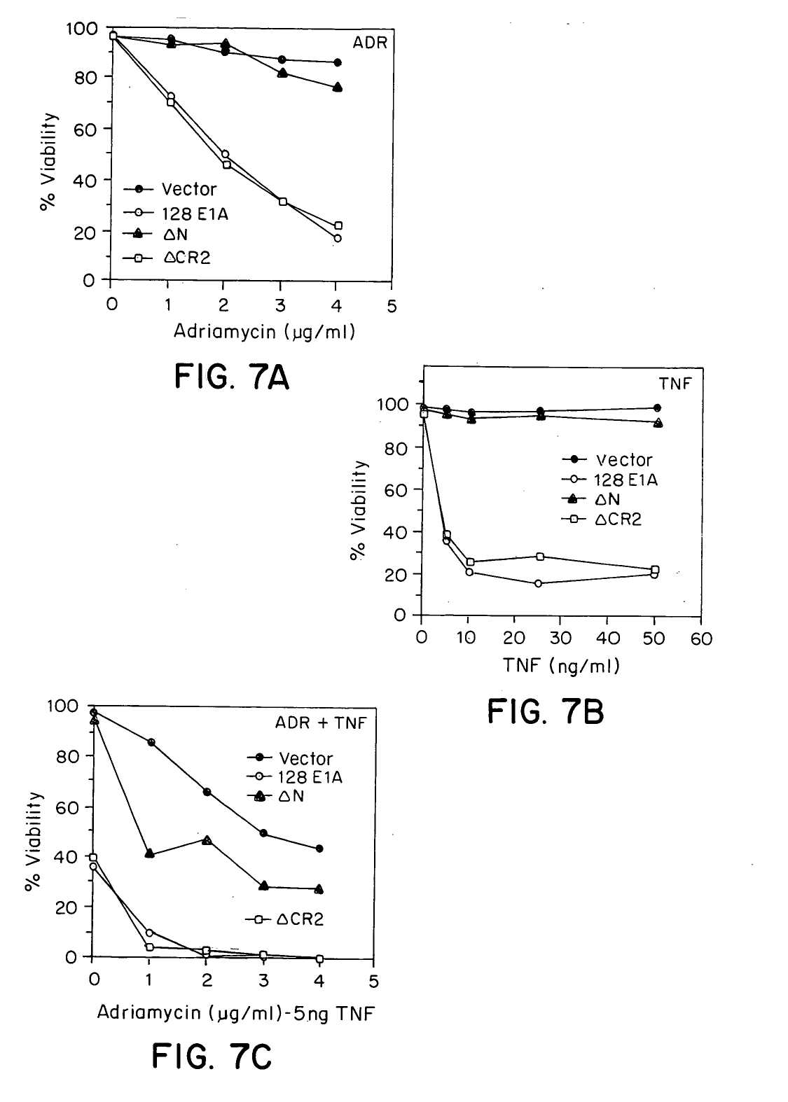

U2OS osterosarcoma cells do not express p16 and hence have defects in the RB pathway. These cells do not undergo apoptosis following treatment with either adriamycin or TNF at the doses tested (closed circles), although the combination produces some cell death. The results, which are shown in FIGS. 7A-7C, demonstrate that E1A (open circles) enhances the cytotoxicity of both ADR and TNF (or the combination). The E1A DN mutant, which retains its ability to bind RB but is unable to enhance chemosensitivity in normal cells is also unable to enhance chemosensitivity in U2OS cells (this is the predicted result). The E1A ΔCR2 (open squares), on E1A mutant which is unable to enhance drug cytotoxicity in normal cells, promotes chemosensivity as efficiently as full length E1A. These data imply that the proposed strategy is effective in tumor lines harboring normal Rb but with other defects in the Rb pathway.

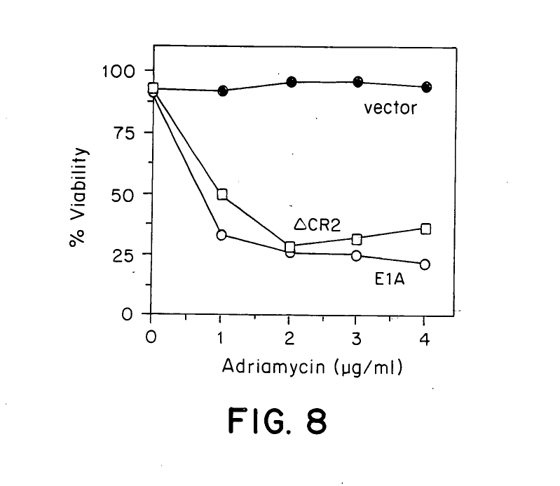

BT-549 breast carcinoma cells have mutant Rb. As shown in FIG. 8, like Saos2 cells, adriamycin is unable to induce apoptosis in BT549 cells containing a control vector (closed circles) but effectively induces cell death in cells expressing full length E1A (open circles). Again the E1A ΔCR2 mutant (open squares) promotes chemosensivity as efficiently as full length E1A.

These data demonstrate that these E1A mutants (or mimics) will selectively enhance the chemosensitivity of tumor cells.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. An E1A mutant which does not bind and inactivate retinoblastoma protein in normal mammalian cells.

2. An E1A mutant of claim 1 which, when expressed in a retinoblastoma protein deficient mammalian cell, enhances sensitivity of the retinoblastoma protein deficient mammalian cell to a chemotherapeutic agent or irradiation.

3. An E1A mutant of claim 2 which lacks conserved region 2 of E1A or a portion thereof.

4. An E1A mutant of claim 3 which lacks amino acid residues 120 to 140 of E1A 12S protein.

5. An E1A mutant of claim 2 wherein at least one amino acid residue of conserved region 1 of E1A 12S and at least one amino acid residue of conserved region 2 of E1A 12S are deleted, modified or replaced.

6. An E1A mutant of claim 5 wherein the tyrosine residue at position 47 of conserved region 1 and the tyrosine residue at position 124 of conserved region 2 are each replaced by an amino acid other than tyrosine.

7. An E1A mutant of claim 6 wherein the tyrosine residue at position 47 and the tyrosine residue at position 124 are each replaced by a histidine residue.

8. DNA encoding an E1A mutant which does not bind and inactivate retinoblastoma protein in normal mammalian cells.

9. DNA of claim 8 encoding an E1A mutant which, when expressed in a retinoblastoma protein deficient mammalian cell, enhances sensitivity of the retinoblastoma protein deficient mammalian cell to a chemotherapeutic agent or irradiation.

10. DNA of claim 9 encoding an E1A mutant which lacks conserved region 2 of E1A or a portion thereof.

11. DNA of claim 10 encoding an E1A mutant which lacks amino acid residues 120 to 140 of E1A 12S protein.

12. DNA of claim 9 encoding an E1A mutant wherein at least one amino acid residue of conserved region 1 of E1A 12S and at least one amino acid residue of conserved region 2 of E1A 12S are deleted, modified or replaced.

13. DNA of claim 12 encoding an E1A mutant wherein the tyrosine residue at position 47 of conserved region 1 and the tyrosine residue at position 124 of conserved region 2 are each replaced by an amino acid other than tyrosine.

14. DNA of claim 13 encoding an E1A mutant wherein the tyrosine residue at position 47 and the tyrosine residue at position 124 are each replaced by a histidine residue.

15. A vector comprising DNA encoding an E1A mutant which does not bind and inactivate retinoblastoma protein in normal mammalian cells, wherein the vector expresses the DNA when present in a mammalian cell.

16. A vector of claim 15 comprising DNA encoding an E1A mutant which, when expressed in a retinoblastoma deficient mammalian cell, enhances sensitivity of the retinoblastoma deficient mammalian cell to a chemotherapeutic agent or irradiation.

17. A vector of claim 16 comprising DNA encoding an E1A mutant which lacks conserved region 2 E1A or a portion thereof.

18. A vector of claim 17 comprising DNA encoding an E1A mutant which lacks amino acid residues 120 to 140 of E1A 12S protein.

19. A vector of claim 16 comprising DNA encoding an E1A mutant wherein at least one amino acid residue of conserved region 1 of E1A 12S and at least one amino acid residue of conserved region 2 of E1A 12S are deleted, modified or replaced.

20. A vector of claim 19 comprising DNA encoding an E1A mutant wherein the tyrosine residue at position 47 of conserved region 1 and the tyrosine residue at position 124 of conserved region 2 are each replaced by an amino acid other than tyrosine.

21. A vector of claim 20 comprising DNA encoding an E1A mutant wherein the tyrosine residue at position 47 and the tyrosine residue at position 124 are each replaced by a histidine residue.

22. A method of enhancing sensitivity of a retinoblastoma deficient mammalian cell to a chemotherapeutic agent or irradiation, comprising introducing into the retinoblastoma deficient mammalian cell an E1A mutant which does not bind and inactivate a retinoblastoma protein in a normal mammalian cell.

23. The method of claim 22 wherein the E1A mutant is an E1A mutant which lacks conserved region 2 of E1A 12S protein or an E1A mutant in which at least one amino acid residue of conserved region 1 of E1A 12S and at least one amino acid residue of conserved region 2 of E1A 12S are deleted, modified or replaced.

24. The method of claim 23 wherein the E1A mutant is an E1A mutant which lacks amino acid residues 120 to 140 of E1A 12S protein or an E1A mutant wherein the tyrosine residue at position 47 of conserved region 1 and the tyrosine residue at position 124 of conserved region 2 are replaced by an amino acid residue other than tyrosine.

25. The method of claim 24 wherein in the E1A mutant, the tyrosine residue at position 47 and the tyrosine residue at position 124 are each replaced by a histidine residue.

26. The method of claim 22 wherein the E1A mutant is introduced into the cell using an adenoviral vector.

27. A method of enhancing sensitivity of a retinoblastoma deficient tumor cell to a chemotherapeutic agent or irradiation in an individual to whom a chem.-therapeutic agent or radiation is being administered, comprising administering to the individual an E1A mutant protein which does not bind and inactivate a retinoblastoma protein in normal cells or DNA encoding an E1A mutant protein which does not bind and inactivate a retinoblastoma protein in normal cells, in such a manner that the E1A mutant protein enters tumor cells or the DNA encoding the E1A mutant protein enters tumor cells and is expressed therein.

28. The method of claim 27, wherein the E1A mutant is an E1A mutant which lacks conserved region 2 of E1A 12S protein or an E1A mutant wherein at least one amino acid residue of conserved region 1 of E1A 12 and at least one amino acid residue of conserved region 2 are deleted, modified or replaced.

29. The method of claim 27 wherein the E1A mutant is administered to the individual using an adenoviral vector.

30. A polypeptide consisting essentially of the amino acid residues of the E1A N-terminus.

31. A molecule or compound which mimics the activity of the E1A N-terminus when introduced into a wild-type mammalian cell and when introduced into a retinoblastoma protein deficient mammalian cell.

32. A molecule or compound of claim 31 wherein the activity of the E1A N-terminus is binding to retinoblastoma protein, p300 protein and CBP.

33. A molecule or compound which mimics the activity of an E1A mutant selected from the group consisting of E1A mutants which lack conserved region 2 of E1A and E1A mutants which have a mutation of at least one amino acid residue in conserved region 1 and a mutation of at least one amino acid residue in conserved region 2, when the E1A mutant is introduced into a wild-type mammalian cell and when the E1A mutant is introduced into a retinoblastoma protein deficient mammalian cell.

34. A molecule of claim 33 wherein the activity of the E1A mutant is binding to retinoblastoma protein, p300 protein and CBP.

35. A method of enhancing sensitivity of a retinoblastoma protein deficient mammalian cell to a chemotherapeutic agent or radiation, comprising introducing into the retinoblastoma protein deficient mammalian cell a polypeptide consisting essentially of the amino acid residue of the E1A N-terminus or a molecule or compound which mimics the activity of the E1A N-terminus when introduced into a wild-type mammalian cell and when introduced into a retinoblastoma protein deficient mammalian cell.

36. The method of claim 35 wherein the activity of the E1A N-terminus is binding to retinoblastoma protein, p300 protein and CBP.

37. The method of claim 35 wherein the polypeptide is introduced into the cell using an adenoviral vector.

38. A method of enhancing sensitivity of a retinoblastoma protein deficient mammalian cell to a chemotherapeutic agent or radiation, comprising introducing into the retinoblastoma protein deficient mammalian cell a molecule or compound which mimics the activity of an E1A mutant, selected from the group consisting of E1A mutants which lack conserved region 2 of E1A and E1A mutants which have a mutation of at least one amino acid residue in conserved region 1 and a mutation of at least one amino acid residue in conserved region 2, when the E1A mutant is introduced into a wild-type mammalian cell and when the E1A mutant is introduced into a retinoblastoma protein deficient mammalian cell.

39. The method of claim 38 wherein the activity of the E1A mutant is binding to retinoblastoma protein, p300 protein and CBP.

40. A method of identifying an E1A mutant which does not bind and inactivate retinoblastoma protein in wild-type mammalian cells and which enhances sensitivity to chemotherapeutic agents and irradiation in retinoblastoma deficient mammalian cells, comprising:
a) expressing an E1A mutant in wild-type mammalian cells and in retinoblastoma deficient mammalian cells of the same type, thereby producing wild-type mammalian cells expressing the E1A mutant and retinoblastoma deficient mammalian cells expressing the E1A mutant;
b) contacting the mammalian cells produced in a) with an agent which stimulates apoptosis in mammalian cells;
c) determining whether apoptosis occurs in the wild-type mammalian cells and in the retinoblastoma deficient mammalian cells; and
d) comparing the extent to which apoptosis occurs in the retinoblastoma deficient mammalian cells to the extent to which apoptosis occurs in wild-type mammalian cells in which full length E1A is expressed which are contacted with the agent which stimulates apoptosis in mammalian cells as in b),
wherein if apoptosis does not occur in the wild-type mammalian cells expressing the E1A mutant or occurs to a lesser extent in the wild-type mammalian cells expressing the E1A mutant than in the retinoblastoma deficient mammalian cells expressing the E1A mutant and apoptosis occurs to a similar extent in the retinoblastoma deficient mammalian cells expressing the E1A mutant and in the wild-type mammalian cells expressing full length E1A, the E1A mutant is an E1A mutant which does not bind and inactivate retinoblastoma protein in wild-type mammalian cells and which enhances sensitivity to chemotherapeutic agents and irradiation in retinoblastoma deficient mammalian cells.

41. A method of identifying a molecule which mimics the function of an E1A mutant wherein the molecule does not bind and inactivate retinoblastoma protein in wild-type mammalian cells and which enhances sensitivity to chemotherapeutic agents and irradiation in retinoblastoma deficient mammalian cells, comprising:
a) contacting retinoblastoma deficient mammalian cells and wild type mammalian cells with a molecule to be assessed and a sublethal dose of an agent which stimulates apoptosis in mammalian cells;
b) determining whether apoptosis occurs in the wild-type mammalian cells and in the retinoblastoma deficient mammalian cells; and
c) comparing the extent to which apoptosis occurs in the retinoblastoma deficient mammalian cells to the extent to which apoptosis occurs in wild-type mammalian cells,
wherein if apoptosis does not occur in the wild-type mammalian cells or occurs to a lesser extent than in the retinoblastoma deficient mammalian cells, then the molecule to be assessed is a molecule which mimics the function of an E1A mutant wherein the molecule does not bind and inactivate retinoblastoma protein in wild-type mammalian cells and enhances sensitivity to chemotherapeutic agents and irradiation in retinoblastoma deficient mammalian cells.

42. The method of claim 41 wherein the retinoblastoma deficient cells are selected from the group consisting of: cells derived from a retinoblastoma knockout mammal and tumor cells.

43. The method of claim 42 wherein the cells derived from a retinoblastoma knockout mammal are mouse embryo fibroblasts derived from a retinoblastoma knockout mouse.

44. The method of claim 42 wherein the tumor cells are selected from the group consisting of: Snos2 osteosarcoma cells, U2OS osteosarcoma cells and BT-549 breast carcinoma cells.
